(19) United States
(12) Patent Application Publication
(10) Pub. No.: US 2020/0037879 A1
(43) Pub. Date: Feb. 6, 2020

(54) WIRELESS MEDICAL IMPLANTS AND METHODS OF USE

(71) Applicant: Integrated Sensing Systems, Incorporated, Ypsilanti, MI (US)

(72) Inventor: Nader Najafi, Ann Arbor, MI (US)

(21) Appl. No.: 16/520,291

(22) Filed: Jul. 23, 2019

Related U.S. Application Data
(60) Provisional application No. 62/764,185, filed on Jul. 23, 2018.

(51) Int. Cl. A61B 5/00; A61B 5/028; A61B 5/029; A61B 5/0215

(52) U.S. Cl. CPC A61B 5/0031; A61B 5/0006; A61B 5/0008; A61B 5/029; A61B 5/0215; A61B 5/028

(57) ABSTRACT

Wireless medical implants and methods for wirelessly monitoring cardiac output (CO) of a subject. Such a method includes placing the wireless medical implant in a wall of an organ of the subject such that an end of the wireless medical implant containing a temperature transducer is exposed to blood flowing in the organ, introducing a substance into the blood flowing at a site so that the substance is at a different temperature than the blood flowing through the organ and the wireless medical implant is downstream of the site where the substance was introduced, and wirelessly and continuously measuring cardiac output by wirelessly and continuously obtaining temperature measurements with the wireless medical implant downstream of the site where the substance was introduced to generate of a thermodilution curve from which reproducible calculations of cardiac output are obtained.

### CROSS-REFERENCE TO RELATED APPLICATIONS

This application claims the benefit of U.S. Provisional Application No. 62/764,185, filed Jul. 23, 2018, the contents of which are incorporated herein by reference.

### BACKGROUND OF THE INVENTION

The present invention generally relates to implantable medical devices, such as of the types for monitoring physiological parameters. The invention particularly relates to methods of measuring cardiac flow parameters (such as cardiac output) and blood flow properties using a wireless medical implant.

Wireless implantable hemodynamic monitors (IHM) have been recently developed, notable examples of which include implantable wireless sensors developed by Integrated Sensing Systems, Inc. (ISS). These sensors are adapted for the measurement of pressure parameters such as mean pulmonary artery, left heart filling pressure (i.e., mean left atrium pressure or left ventricle end diastolic pressure), A-wave, V-wave, peak systolic, heart bit, etc.

Cardiac output (CO, also denoted by the symbols Q and Qc) is a term used in cardiac physiology to describe the volume of blood being pumped by the heart, in particular by the left or right ventricle, per unit time, such as dm³/min and L/min. Because cardiac output is related to the quantity of blood delivered to various parts of the body, it is an important indicator of how efficiently the heart can meet the demands of the body. For instance, infections are correlated with high CO values and heart failures are correlated with low CO values. Along with stroke volume (SV), cardiac output is a global blood flow parameter of interest in hemodynamics—the study of the flow of blood under external forces. The factors affecting stroke volume and heart rate also affect cardiac output. Related measurements include ejection fraction, cardiac input, cardiac index, and combined cardiac output.

There are many methods of measuring CO, both invasively and noninvasively, each with its own advantages and drawbacks. CO measurement techniques include Doppler ultrasound, pulse pressure methods, impedance cardiography, ultrasound dilution, electrical cardiometry, magnetic resonance imaging, the Fick principle, and pulmonary artery thermodilution (trans-right-heart thermodilution). Pulmonary artery thermodilution involves the measurement of temperature changes at sites in a subject's circulation system. The pulmonary artery catheter (PAC), also known as the Swan-Ganz catheter, provides direct access to the right heart for thermodilution measurements. Though continuous, invasive, cardiac monitoring in intensive care units has been largely phased out, the PAC remains useful in right-heart study done in cardiac catheterization laboratories.

Thermodilution methods utilizing a PAC involve inflating the balloon tip of the PAC to assist in delivering the catheter through the right ventricle to occlude a small branch of the pulmonary artery system. After the balloon is deflated, a small amount (for example, 10 ml) of a fluid (e.g., saline or glucose) at a known temperature (below body temperature) is injected into the pulmonary artery and the temperature of blood flowing through the pulmonary artery is measured a known distance downstream of the injection site (e.g., about 6 to 10 cm) using temperature sensors (for example, thermistors) placed on the PAC and set apart from each other at predetermined intervals.

Thermodilution methods of the type described above have historically allowed reproducible calculations of cardiac output from a measured time-temperature curve, also known as the thermodilution curve. Slow temperature changes are indicative of low CO and more rapid temperature changes are indicative of higher CO, and therefore the degree of temperature change sensed in a series of temperature sensors is directly proportional to the cardiac output. A catheter can also be fitted with a heating filament that intermittently heats that blood, such that the resulting thermodilution curve can provide serial Q measurements. Depending on the stability of the circulation, three or four repeated measurements or passes performed over a period of minutes may be averaged to improve accuracy.

PAC thermodilution methods do not allow for continuous monitoring of CO, and require that the patient is in the supine position. Moreover, PAC use can be complicated by arrhythmia, infection, pulmonary artery rupture and damage to the right heart valve. Recent studies in patients with critical illnesses, sepsis, acute respiratory failure and heart failure suggest that use of the PAC does not improve patient outcomes. Clinical ineffectiveness may relate to its poor accuracy and sensitivity, which have been demonstrated by comparison with flow probes across a six-fold range of Q values. As a result, the use of PAC is in decline as clinicians move to less invasive and more accurate technologies for monitoring hemodynamics. Geerts et al., Methods of Pharmacology Measurement of Cardiac Output, Br J Clin Pharmacol, 71:3 (2011) p316-330, provides an overview of CO measurement and techniques therefor.

### BRIEF SUMMARY OF THE INVENTION

The present invention provides wireless medical implants and methods for wirelessly monitoring cardiac output (CO) of a subject.

According to one aspect of the invention, a method is provided for measuring cardiac output of a subject using a wireless medical implant. The method includes placing the wireless medical implant in a wall of an organ of the subject such that an end of the wireless medical implant containing a temperature transducer is exposed to blood flowing in the organ, introducing a substance into the blood flowing at a site so that the substance is at a different temperature than the blood flowing through the organ and the wireless medical implant is downstream of the site where the substance was introduced, and wirelessly and continuously measuring cardiac output by wirelessly and continuously obtaining temperature measurements with the wireless medical implant downstream of the site where the substance was introduced to generate of a thermodilution curve from which reproducible calculations of cardiac output are obtained.

Technical aspects of methods as described above preferably include the ability to wirelessly measure cardiac output of a subject's heart by wirelessly measuring the temperature of blood flowing through an organ of the subject over extended periods of time, with or without other sensed parameters, such as during medical procedures, home monitoring, office visits, and hospital stays to provide indications of the subject's health and/or the effectiveness of medical treatment (e.g., medication, hardware, etc.).

Other aspects and advantages of this invention will be appreciated from the following detailed description.

### BRIEF DESCRIPTION OF THE SEVERAL VIEWS OF THE DRAWING

FIG. 1 is a perspective view of a wireless medical implant comprising a sensing device mounted to an anchor, with the anchor being portrayed in a deployed configuration in accordance with a nonlimiting embodiment of this invention.

FIG. 2 schematically represents a perspective view of a bolt-type anchor capable of use with a sensing device of the type represented in FIG. 1 in accordance with another nonlimiting embodiment of the invention.

FIG. 3 is a proximal end view of the bolt-type anchor of FIG. 2, FIG. 4 is a cross-sectional view taken along line 4-4 in FIG. 3, FIG. 5 schematically represents a manner in which the bolt-type anchor of FIGS. 2 through 4 can be assembled with a sensing device to form a wireless medical implant, and FIG. 6 schematically represents the resulting medical implant.

### DETAILED DESCRIPTION OF THE INVENTION

Nonlimiting embodiments of the invention disclosed herein include measurement of cardiac flow parameters (such as cardiac output), and optionally also blood flow properties such as oxygen content and delivery. The embodiments also allow for measurement of other important cardiac parameters, such as blood flow indicators (for example, Cardiac Index (CI), Continuous Cardiac Index (PCCI), and Continuous Stroke Volume Index (SVI)), Preload/Volume responsiveness indicators (for example, Global End-diastolic Volume Index (GEDI), Stroke Volume Variation (SVV), and Pulse Pressure Variation (PPV)), afterload indicators (for example, System Vascular Resistance Index (SVRI)), contractility indicators (for example, Global Ejection Fraction (GEF), Cardiac Function Index (CFI), Systolic Pressure Increase (dpmx), and Cardiac Power Index (CPI)), and lung function indicators (for example, Extravascular Lung Water Index (ELWI) and Pulmonary Capillary Permeability Index (PVPI)).

In certain embodiments of the invention, a wireless medical implant comprises one or more temperature sensors, while other embodiments alternatively or additionally comprise one or more pressure sensors, ultrasonic sensors, acoustic sensors, or other types of parameter sensors to provide for multiple parameter sensing capabilities. The implants preferably make use of an anchor that enables the implants to remain implanted in a subject for long durations.

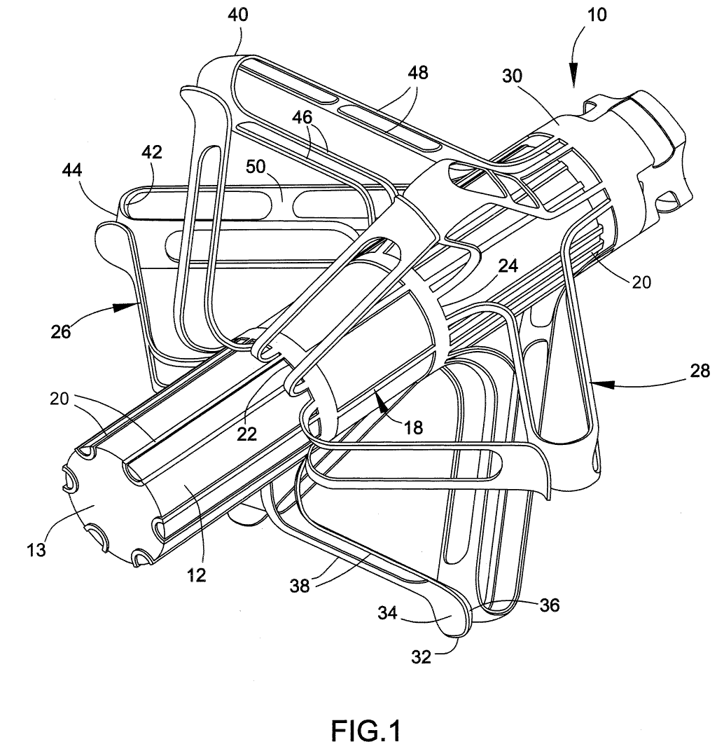

FIG. 1 depicts an implantable unit comprising an anchor 10 suitable for delivering and securing a wireless medical implant 12 to a wall of an internal organ in accordance with an embodiment of the present invention. The implant 12 may be, but is not limited to, one of a type disclosed in U.S. Pat. Nos. 8,744,544, 8,715,300, 8,696,693, 8,512,252, 8,322,346, 8,267,863, 8,014,865, 7,860,579, 7,686,762, 7,634,319, 7,615,010, 7,317,951, and 6,968,743, whose contents are incorporated herein by reference. In FIG. 1, the implant 12 is represented as having a cylindrical shape defined by a housing that contains a temperature sensing element/transducer, at least one inductor coil for wirelessly (telemetrically) communicating (both reception and transmission) with an external reader unit (not shown), and electronics for signal conversion and communication. Though the implant 12 may be equipped with a battery, in preferred embodiments the energy required to operate the implant 12 is entirely derived from the reader unit. One end 13 of the implant 12 preferably serves as the location of the transducer, such as a temperature sensor (e.g., thermistor). Ideally, the implant 12 is of minimal size, a nonlimiting example being a diameter of about 3.7 millimeters and a length of about 10 millimeters. While a cylindrical implant 12 is shown in FIG. 1, the functionality of the anchor 10 is not dependent on any particular shape for the implant 12, and can be readily adapted to secure a variety of different shaped implants with different sensing technologies.

The anchor 10 is configured to be placed with a positioning catheter (not shown). The anchor 10 is depicted in FIG. 1 in what will be termed a deployed configuration, meaning the configuration of the anchor 10 when placed in a wall of an internal organ (for example, heart, vein, artery, aneurysm sac, etc.), so that at least the end of the implant 12 containing the transducer is exposed to blood flowing in the organ. The anchor 10 is shown as having an annular-shaped base portion 18 that surrounds the implant 12. The base portion 18 is represented as having a frame-like construction that defines a cage 20 in which the implant 12 is located. The base portion 18 has oppositely-disposed first and second ends 22 and 24 corresponding to oppositely-disposed first and second longitudinal directions parallel to a central axis of the base portion 18, which also defines a longitudinal axis of the anchor 10. For convenience, these directions will be referred to as distal and proximal directions, and various structures of the anchor 10, including the ends 22 and 24 of the base portion 18, will be described as being distal or proximal to reflect the orientation of the anchor 10 during an implantation procedure. However, it should be understood that the invention is not necessarily limited to any particular orientation for the anchor 10.

When stowed, structures of the anchor 10, referred to as arms 26 and legs 28, extend substantially parallel to the axis of the base portion 18 from its distal and proximal ends 22 and 24, respectively. The legs 28 support an annular-shaped coupler member 30, so that the coupler member 30 is axially spaced from the second end 24 of the base portion 18. The arms 26 and legs 28 are resiliently biased so that, when deployed as shown in FIG. 1, the arms 26 and legs 28 acquire shapes that preferably lie within angularly spaced radial planes, each containing the axis of the base portion 18. The deployed arms 26 generally deploy by rotating about their respective attachments to the base portion 18 at the distal end 22 thereof, with movement of the arms 26 generally occurring in the proximal direction so that the arms 26 project substantially radially from the longitudinal axis of the anchor 10. When fully deployed, the arms 26 also extend in the proximal direction relative to the distal end 22 of the base portion 18. Each arm 26 terminates with an extremity or distal tip 32, which in the deployed configuration is radially offset from the longitudinal axis of the anchor 10. The distal tip 32 is represented as having a semispherical shape, such that oppositely-disposed concave and convex surfaces 34 and 36 are defined. With the arms 26 in the deployed configuration, the concave surfaces 34 face the distal direction and the convex surfaces 36 face the proximal direction. Each arm 26 is further shown as comprising beams 38, which are generally parallel to each other and spaced apart from each other in directions transverse to the longitudinal axis of the anchor 10. The beams 38 define spanning portions of the arms 26 that interconnect the distal tips 32 of the arms 26 to the base portion 18. By providing multiple beams 38 within each spanning portion of each arm 26, a level of redundancy is provided in the event one of the beams 38 becomes damaged or breaks.

Each deployed leg 28 generally deploys by rotating about its respective attachment to the base portion 18 at the proximal end 24 thereof, with movement of each leg 28 generally occurring in the distal direction so that the legs 28 project substantially radially from the longitudinal axis of the anchor 10. When fully deployed, the legs 28 also extend in the distal direction (opposite that of the arms 26) relative to the proximal end 24 of the base portion 18. Each leg 28 has an intermediate portion 40, which in the deployed configuration is radially offset from the longitudinal axis of the anchor 10. Similar to the distal tips 32 of the arms 26, each intermediate portion 40 is represented as having a semispherical shape, such that oppositely-disposed concave and convex surfaces 42 and 44 are defined. With the legs 28 in the deployed configuration, the convex surfaces 44 predominantly face the distal direction so as to oppose the distal tips 32 of the arms 26. With the arms 26 and legs 28 in their deployed configurations, the convex surfaces 36 and 44 of the arms 26 and legs 28 are axially aligned with each other, providing a clamping capability on the wall of an organ. Each leg 28 is further shown as comprising two sets of beams 46 and 48. One set of beams 46 is disposed between the proximal end 24 of the base portion 18 and the intermediate portion 40, while the second set of beams 48 is disposed between the coupler member 30 and the intermediate portion 40. As with the beams 38 of the arms 26, the leg beams 46 and 48 are generally parallel to each other and the beams 46 and 48 of each set are spaced apart from each other in directions transverse to the longitudinal axis of the anchor 10. The beams 46 and 48 define spanning portions of the legs 28 that interconnect their intermediate portions 40 to the base portion 18 and coupler member 30, respectively. Again, a benefit of this construction is the ability to provide a level of redundancy in the event one of the beams 46 and 48 becomes damaged or breaks. The legs 28 further include struts 50 that span the gaps between the individual sets of beams 46 and 48, thereby reinforcing the legs 28 and inhibiting any tendency for the legs 28 to twist during deployment.

Other aspects of the anchor 10 can be appreciated from U.S. Pat. Nos. 8,715,300 and 9,468,408, whose contents are incorporated herein by reference.

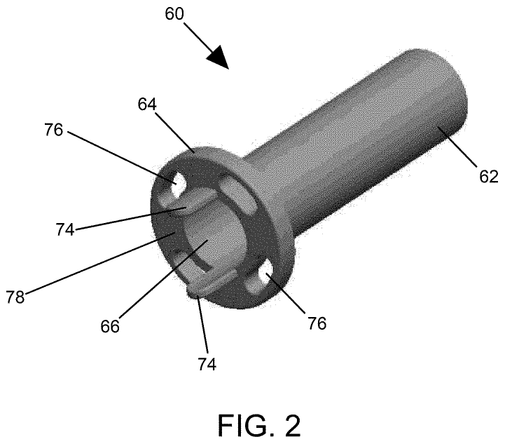

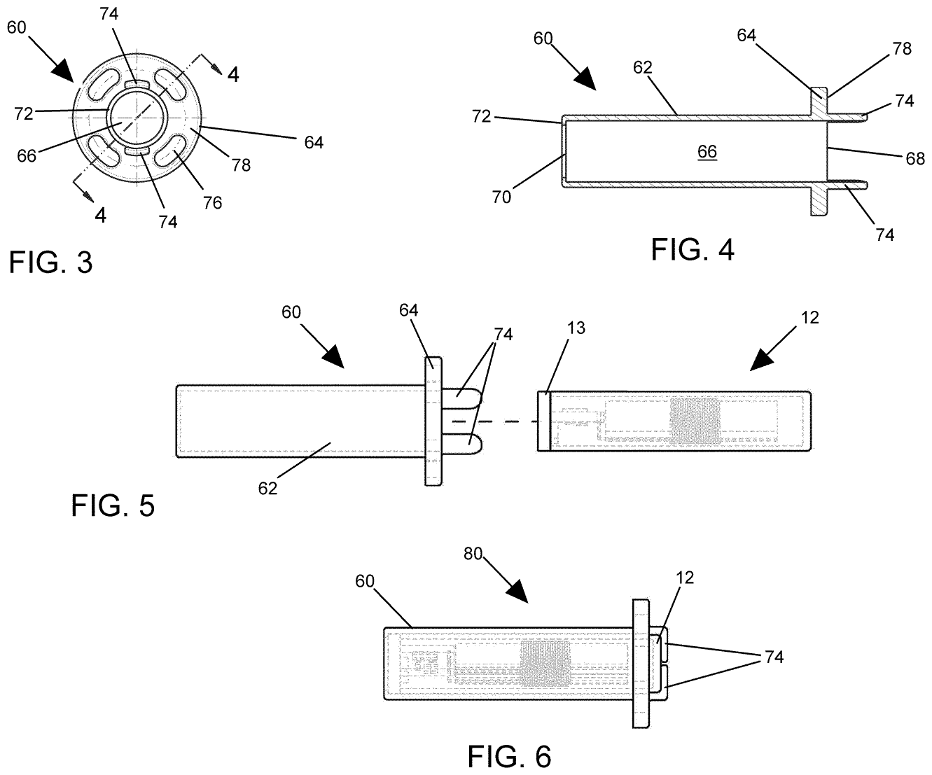

FIGS. 2 through 6 show a bolt-type anchor 60 adapted to be placed in a wall of an organ. In contrast to the anchor 10 of FIG. 1, access to an organ and implantation of the anchor 60 is preferably achieved using an endoscope, for example, via laparoscopic surgery, thoracoscopic surgery, or another similar minimally-invasive procedure, as opposed to translumenal implantation techniques that use a placement catheter to place an implant within an organ and then secure the implant to a wall of the organ. The embodiment of the anchor 60 shown in FIGS. 2 through 6 has a tubular portion 62 and a disk-shaped portion 64 at oppositely-disposed distal and proximal ends, respectively, of the anchor 60, and an internal passage 66 sized to accommodate at least a portion of a sensing device, such as the implant 12 of FIG. 1. The passage 66 preferably has a shape that is complementary or otherwise corresponds to the outer shape of an implant intended to be placed therein, for example, a cylindrical shape corresponding to the cylindrical outer shape of the implant 12, though passages and implants of other and even different shapes are also within the scope of the invention. In the particular embodiment shown in FIGS. 2-6, a proximal portion of the passage 66 defines a proximal opening 68 at a proximal surface 78 of the disk-shaped portion 64, and a distal portion of the passage 66 within the tubular portion 62 defines a distal opening 70 at the distal end of the anchor 60. Also in the illustrated embodiment, the distal opening 70 is configured for retaining the implant 12 within the passage 66, and the proximal opening 68 is sized to enable the implant 12 to pass therethrough into the passage 66 until the implant 12 abuts a feature 72 at the distal opening 70. As a nonlimiting example, the proximal and distal openings 68 and 70 represented in FIGS. 2-6 are both circular in shape, and the distal opening 70 is smaller than the proximal opening 68 as a result of the feature 72 being in the form of a radially inward-extending peripheral lip or rim that surrounds the distal opening 70. It is also within the scope of the invention that the feature 72 (or multiple features) could take other forms, for example, as a result of the distal opening 70 being sized to create an interference fit with the implant 12, one or more flanges or tabs that extend radially inward over the distal opening 70 of the passage 66, an adhesive bond formed with a biocompatible epoxy, glue, or cement, etc.

As represented in FIG. 5, inserting the implant 12 into the tubular portion 62 through the disk-shaped portion 64, and therefore through the proximal end of the anchor 60, yields an implantable unit 80 represented in FIG. 6. The implant 12 is shown as being further secured within the tubular portion 62 by one or more features 74 disposed on the disk-shaped portion 64. Depending on the particular feature 72 provided at the distal opening 70 of the anchor 60, such a feature 74 could be the result of the disk-shaped portion 64 closing the proximal portion of the passage 66, in which case the implant 12 would be inserted into the tubular portion 62 through the distal opening 70 of the anchor 60. On the other hand, the nonlimiting embodiment of FIGS. 2-6 represents the feature 74 as a diametrically-opposed pair of flanges, tabs, or “ears” disposed on the disk-shaped portion 64, which are shown in FIGS. 2, 4 and 5 as originally extending from the proximal surface 78 of the disk-shaped portion 64 in an axial direction of the tubular portion 62. By plastically bending or otherwise deforming the features 74 toward each other after the implant 12 is placed in the passage 66 through the proximal opening 68, the features 74 are able to secure the implant 12 within the passage 66 of the tubular portion 62 by extending radially inward over the proximal opening 68 of the passage 66, thereby capturing the implant 12 between the features 72 and 74 at the distal and proximal ends of the anchor 60. The entire anchor 60 or at least the features 74 thereof can be fabricated from various materials that are capable of contributing the desired plastic deformability of the features 74, a nonlimiting example of which is PEEK.

The implantable unit 80 can be placed in a wall of an internal organ (e.g., heart, artery, aneurysm sac, etc.) and secured thereto, for example, with sutures that pass through multiple openings 76 that are defined in the disk-shaped portion 64 of the anchor 60. The tubular portion 62 of the anchor 60 may be placed within and passes at least partially through a wall (for example, the endocardium lining a chamber of the heart), while the disk-shaped portion 64, which surrounds and projects radially from the tubular portion 62, remains outside the wall and abuts a surface of the wall. In the nonlimiting example of FIGS. 2-6, the tubular portion 62 has an outer cylindrical shape that may facilitate implantation of the anchor 60 and occlusion of an opening in which the unit 80 is placed. For this reason, the tubular portion 62 also preferably defines a continuous annular-shaped wall that entirely surrounds the distal portion of the passage 66 therein, so that the passage 66 is entirely closed except for its proximal and distal openings 68 and 70. The length of the tubular portion 62 can be selected based on the thickness of the wall in which the unit 80 is to be placed, and based on whether the distal end of the unit 80 defined by the tubular portion 62 is intended to protrude beyond the surface of the wall. The distal end of the unit 80 (i.e., the lefthand end of the anchor 60 in FIG. 6) may protrude from the wall surface, for example, not more than one centimeter, and preferably not more than eight millimeters. Alternatively, the unit 80 may be placed in a wall so as not to protrude beyond its surface, for example, the distal end of the unit 80 may be recessed within the wall, for example, up to about two millimeters from its surface. As a result of assembling the anchor 60 and implant 12 in the manner shown in FIGS. 5 and 6, the end 13 of the implant 12 that carries the transducer need not protrude from the passage 66 of the anchor 60, yet is exposed within the distal opening 70 of the anchor 60, such that the distal end of the anchor 60 protrudes farther into the organ than the implant 12 by a distance defined by the axial dimension of the feature 72.

Other aspects of the anchor 60 can be realized from U.S. Patent Application Publication Nos. 2016/0183842 and 2017/0095210, whose contents are incorporated herein by reference.

As previously noted, a nonlimiting aspect of the invention pertains to the use of one or more wireless medical implants placed within the cardiovascular system or in its vicinity to measure temperature for the purpose of wirelessly monitoring cardiac output (CO) of a subject, in which case the transducer located at the end 13 of the implant 12 of any one of FIGS. 1 through 6 is a temperature sensor, as a nonlimiting example, a thermistor. The implant and its anchor (such as described above in reference to FIGS. 1 through 6) may be located, as nonlimiting examples, at or in any one or more of the four chambers of the heart as well as various different veins or arteries of the circulation system. More than one implant may be placed inside a single patient, for example, placed at different locations in the wall of the organ, including farther downstream of the site where the substance was introduced, to obtain additional temperature measurements and thereby provide more accurate data or provide additional information. These implants can all be configured to measure temperature or configured to measure different parameters or multiple parameters. Such additional sensing capabilities include, but are not limited to, pressure sensors, oxygen content sensors, impedance sensors, acoustic sensors, light sensors, infrared sensors (IR) sensors, chemical sensors, gas content sensors, blood sensors/analyzers, ECG, EKG, flow meters, additional temperature sensors, heaters, electrodes, pacing electrodes, etc.

The ability to wirelessly measure temperature within a subject, with or without other sensed parameters, offers important functionalities and benefits. For example, regular monitoring of a subject's temperature over time (through home monitoring or during office visits or hospital stays) provide a useful trend for monitoring the subject's health and/or the effectiveness of medical treatment (e.g., medication, hardware, etc.). Temperature measurements can also assist in the detection of other indications such as infection, inflammation, change of blood flow, or other diseases. However, an aspect of the present invention is to place an implant with a temperature-sensing capability in a subject to wirelessly and continuously measure cardiac output and optionally other associated parameters based on the thermodilution technique previously described. In particular, the implant is placed in an organ of the circulation system of a subject so that the transducer of the implant is exposed to blood flowing in the organ and temperature measurements can be wirelessly and continuously obtained downstream of the site where a relatively cool fluid (e.g., saline, glucose, or other substance) has been introduced into a subject's circulation system, to enable the generation of a thermodilution curve from which reproducible calculations of cardiac output can be obtained, wherein a slow temperature change is indicative of low CO and a more rapid temperature change is indicative of higher CO, such that the degree of temperature change sensed in a series of implants is directly proportional to the cardiac output of a subject's heart.

The wireless medical implants enable the measurement of cardiac output and all of its associated parameters by replacing previous PAC systems and their temperature sensors. Certain techniques that are used with PAC systems can be applied with the implants, but with better performance and fewer problems. The implant can be placed in locations that are superior to where PAC temperature sensors can be placed. For example, the implant can provide a much shorter path between where a cold fluid (e.g., glucose, saline, or other substance) is introduced and where temperature is measured downstream, resulting in a more accurate measurement. Another advantage is that after the implant is placed, it can be noninvasively operated and wirelessly powered and interrogated with an external reading unit with much lower risk than repeating a heart catheterization. Generally, the invasive nature of PAC or other catheter approaches makes them high risk, high cost, and inconvenient for multiple uses on a subject. The use of a wireless medical implant is also less dependent on the proficiency of the operator and readings can be obtained faster and with more accuracy.

Another advantage of measuring cardiac output by measuring temperatures with a wireless medical implant is that the temperature sensing implant can be read with the subject in different positions, such as supine, seating, or standing. Furthermore, the patient can be monitored while performing other activities, such as different levels of exercise. Wireless implants of the type described herein and disclosed in U.S. Pat. Nos. 8,744,544, 8,715,300, 8,696,693, 8,512,252, 8,322,346, 8,267,863, 8,014,865, 7,860,579, 7,686,762, 7,634,319, 7,615,010, 7,317,951, and 6,968,743, have the additional advantage of requiring a fewer number of calibrations, and each calibration is expected to remain useful for longer periods of time since calibration can be performed for the specific subject and his/her specific cardiovascular system.

As previously noted, the wireless medical implants described above can be used in combination with other sensing technologies to measure cardiac output and other associated parameters. Such sensing technologies include but are not limited to pressure sensing transducers and implants (a subset of which is also known as implantable hemodynamic monitors, or IHM) that utilize pressure waveforms to estimate cardiac output and other parameters based on pertinent models, for example, as described in Geerts et al. Other sensing technologies include, but are not limited to, ultrasonic, acoustic, or impedance implants or combinations thereof to measure CO and associated parameters. Such sensing technologies may be implemented as wireless medical implants, similar to what has been described above for the implant 12 and anchors 10 and 60 illustrated in FIGS. 1 through 6, and therefore offers the same or similar advantages described above for wireless medical implants equipped with temperature transducers.

The use of a wireless medical implant that provides extracorporeal acoustic measurements may be particularly advantageous for assisting with the operation and monitoring of cardiac assist devices (such as a left ventricle assist device, or LVAD). Such an implant can be placed as a separate implant that works independently of (but may communicate with) a cardiac assist device, or may be an integrated part of a cardiac assist device. Such an acoustic-sensing implant may offer both improved operation (e.g., adjusting the pump speed) and improved safety, for example detection of LVAD malfunction or thrombogenicity issues. In addition or alternatively, an acoustic-sensing implant may be used to monitor the health of and changes in the heart over time. For example, the progression of congestive heart failure or the effectiveness of a medication can be monitored by regular monitoring of acoustic measurements. Different types of cardiac diseases can be monitored by acoustic measurement over time, in particular mitral regurgitation and arrhythmia (such atrial fluttering and fibrillation).

One or more wireless medical implants that provide combinations of pressure waveform (hemodynamics) and acoustic measurements, enabling acoustic samples (for example, over a period of ten seconds) to be analyzed and the results presented to a medical staff. This analysis could include absolute values as of the time of sampling or a comparison to the past (or a baseline) in order to depict the changes in the state of a patient and their treatment. The analysis could be in the time domain or frequency domain or a combination thereof. Certain frequencies may be chosen for trend charts to monitor the patient and their treatment and/or medication, or monitor a medical device implanted in the patient, or a combination thereof.

In view of the foregoing, wireless medical implants of the types described above can be utilized in a wide variety of settings, including pre-operative preparations, during an operation, during post op, within an intensive care unit (ICU), during a hospital stay, during an emergency visit, during a doctor visit, and during home monitoring. The implants and their use provide for long-term advanced monitoring of a variety of subjects and conditions, including shock of any cause, post-operative management of unstable intensive care patients, diagnosis of pulmonary edema in critically ill patients, early goal directed therapy of patients in shock, peri-operative monitoring of high risk patients and/or high risk interventions, and perioperative goal-directed therapy. Benefits to such patients may include shorter time on mechanical ventilation, shorter ICU stays, sooner ICU discharges, less volume loading and better patient outcomes, lower dosages and shorter durations of vasopressors and catecholamines, fewer neurological complications such as vasospasm, delayed ischemic neurological deficits, cerebral infarction, and neurological deficits, fewer organ failures including renal insufficiency, improved outcomes of pediatric burn patients, and reductions of incidence of acute kidney injury (AKI).

While the invention has been described in terms of specific embodiments, it is apparent that other forms could be adopted by one skilled in the art. As nonlimiting examples, the configurations of the implants and anchors could differ from what those depicted in the drawings, functions of certain components of the implants and anchors could be performed by components of different construction but capable of a similar (though not necessarily equivalent) function, and various materials could be used in the fabrication of the implants, anchors, and/or their components. As such, it should be understood that the above detailed description is intended to describe the particular embodiments represented in the drawings and certain but not necessarily all features and aspects thereof, and to identify certain but not necessarily all alternatives to the represented embodiments and their described features and aspects. As a nonlimiting example, the invention encompasses additional or alternative embodiments in which one or more features or aspects of a particular embodiment could be eliminated or two or more features or aspects of different embodiments could be combined. Accordingly, it should be understood that the invention is not necessarily limited to any embodiment described herein or illustrated in the drawings, and the phraseology and terminology employed above are for the purpose of describing the illustrated embodiments and do not necessarily serve as limitations to the scope of the invention. Therefore, the scope of the invention is to be limited only by the following claims.

1. A method of measuring cardiac output of a subject using a wireless medical implant, the method comprising:
placing the wireless medical implant in a wall of an organ of the subject such that an end of the wireless medical implant containing a temperature transducer is exposed to blood flowing in the organ;
introducing a substance into the blood flowing at a site so that the wireless medical implant is downstream of the site where the substance was introduced, the substance being at a different temperature than the blood flowing through the organ; and
wirelessly and continuously measuring cardiac output by wirelessly and continuously obtaining temperature measurements with the wireless medical implant downstream of the site where the substance was introduced to generate of a thermodilution curve from which reproducible calculations of cardiac output are obtained.

2. The method according to claim 1, further comprising telemetrically communicating with the wireless medical implant to obtain a reading of the temperature measurements.

3. The method according to claim 1, wherein the organ is chosen from the group consisting of a chamber of the heart, a vein, or an artery of the circulation system of the subject.

4. The method according to claim 1, wherein the method comprises:
placing at least a second of the wireless medical implant in the wall of the organ farther downstream of the site where the substance was introduced; and
obtaining additional temperature measurements with the second wireless medical implant.

5. The method according to claim 1, wherein the subject is in a supine, seated, or standing position during the method.

6. The method according to claim 1, wherein the placing step comprises:
assembling the wireless medical implant with an anchor;
using the anchor to position at least part of a portion of the wireless medical implant within the wall of the organ;
abutting a disk-shaped portion of the anchor against a surface of the wall of the organ; and
suturing the disk-shaped portion of the anchor to the wall of the organ to secure the wireless medical implant within the wall of the organ.

7. The method according to claim 6, wherein the tubular and disk-shaped portions of the anchor do not clamp the wall therebetween.

8. The method according to claim 6, wherein the anchor and the wireless medical implant are placed using a minimally-invasive procedure.

9. The method according to claim 1, wherein the placing step comprises:
assembling the wireless medical implant with an anchor;
using the anchor to position at least part of a portion of the wireless medical implant within the wall of the organ; and
deploying arms and legs of the anchor on opposite sides of the wall of the organ to secure the wireless medical implant within the wall of the organ.

10. The method according to claim 9, wherein the arms and legs of the anchor clamp the wall therebetween.

11. The method according to claim 9, wherein the anchor and the wireless medical implant are placed using a catheter.

12. The method according to claim 1, wherein the wireless medical implant or a second wireless medical implant additionally measures at least one physiological parameter chosen from the group consisting of pressure, acoustics, and oxygen content of the blood flowing through the organ.

13. The method according to claim 1, wherein the wireless medical implant or a second wireless medical implant additionally measures pressure of the blood flowing through the organ.

14. The method according to claim 1, wherein the method utilizes at least a second wireless medical implant that measures pressure of the blood flowing through the organ.

15. The method according to claim 1, wherein the wireless medical implant or a second wireless medical implant additionally measures acoustics of the blood flowing through the organ.

16. The method according to claim 1, wherein the method utilizes at least a second wireless medical implant that measures acoustics of the blood flowing through the organ.

17. The method according to claim 1, wherein the method utilizes at least a second wireless medical implant chosen from the group consisting of a pressure sensor, oxygen content sensor, impedance sensor, acoustic sensor, ultrasonic sensor, light sensor, infrared sensors (IR) sensor, chemical sensor, gas content sensor, blood sensor/analyzer, ECG, EKG, flow meter, heater, electrode, or pacing electrode.

18. The method according to claim 1, wherein the method is performed during at least one of the following medical settings: a pre-operative preparation, during an operation, during post op, within an intensive care unit, during a hospital stay, during an emergency visit, and during a doctor visit.

20. A method of measuring cardiac output of a subject using a wireless medical implant, the method comprising:
assembling the wireless medical implant with an anchor;
using the anchor to position at least part of a portion of the wireless medical implant in a wall of an organ of the subject such that an end of the wireless medical implant containing a temperature transducer is exposed to blood flowing in the organ;
introducing a substance into the blood flowing at a site so that the wireless medical implant is downstream of the site where the substance was introduced, the substance being at a lower temperature than the blood flowing through the organ; and
wirelessly and continuously measuring cardiac output by wirelessly and continuously obtaining temperature measurements with the wireless medical implant downstream of the site where the substance was introduced to generate of a thermodilution curve from which reproducible calculations of cardiac output are obtained.
